(19) United States
(12) Patent Application Publication
(10) Pub. No.: US 2025/0318897 A1
(43) Pub. Date: Oct. 16, 2025

(54) GAUGE APPARATUS FOR SURGICAL TOOL

(71) Applicant: Arthrex, Inc., Naples, FL (US)

(72) Inventors: Brian Chen, Gainesville, FL (US); Rayan Rupesh Dharia, Gainesville, FL (US); Daniel Giraldo, Gainesville, FL (US); Kellen McKenney, Gainesville, FL (US); Andrea Kannita Noy, Gainesville, FL (US); Conrad Wyrick, Gainesville, FL (US); Kurt Schulze, Gainesville, FL (US); Alexander Hild, Naples, FL (US); Elizabeth Wildsmith, Naples, FL (US)

(73) Assignee: Arthrex, Inc., Naples, FL (US)

(21) Appl. No.: 19/098,293

(22) Filed: Apr. 2, 2025

Related U.S. Application Data
(60) Provisional application No. 63/632,733, filed on Apr. 11, 2024.

(51) Int. Cl. A61B 90/00

(52) U.S. Cl. CPC A61B 90/06; A61B 2090/062

(57) ABSTRACT

A gauge apparatus for a surgical tool includes an orientation sensor configured to detect orientation data identifying a device orientation of the surgical tool about a plurality of axes. A depth sensor measures depth data indicating a tool depth along a longitudinal axis of a tool accessory. A display device provides feedback regarding the device orientation and tool depth. A controller tracks and displays the device orientation and the tool depth of the surgical tool relative to a first offset selectively programmed to a first offset.

### CROSS-REFERENCE TO RELATED APPLICATIONS

This application claims priority under 35 U.S.C. § 119 (e) and the benefit of U.S. Provisional Application No. 63/632,733 entitled GAUGE APPARATUS FOR SURGICAL TOOL, filed on Apr. 11, 2024, by Brian Chen et al., the entire disclosure of which is incorporated herein by reference.

### BACKGROUND

The present disclosure generally relates to a gauge apparatus for a surgical tool and, more particularly, to a depth and orientation measurement device for a surgical driving or cutting tool. As provided in the following disclosure, the use of various surgical tools in various procedures may rely on maneuvering by medical professionals to accurately execute a surgical plan. The disclosure provides for a gauge apparatus that may assist in orienting surgical tools for improved operation and patient outcomes.

### SUMMARY

The disclosure provides for a gauge apparatus for a surgical hand tool that may be implemented to provide intraoperative guidance to a user to ensure that an orientation of a surgical tool is consistently aligned with the target region. In various implementations, the gauge apparatus may correspond to a detachable accessory that may be selectively implemented depending on the specific application of the surgical tool and a corresponding tool accessory applied to conduct a procedure. For example, the tool accessory may correspond to a cutting tool or a driving tool that may be implemented to manipulate or resect tissue or bone of a patient according to a preoperative plan. In various implementations, the gauge apparatus may incorporate an orientation sensor configured to detect an orientation of the surgical tool about a plurality of axes. Additionally, the gauge apparatus may incorporate a depth sensor having a detection field extending along a longitudinal axis of the tool accessory. In this configuration, the gauge apparatus may be applied to monitor the device orientation and a depth of the tool accessory and provide feedback to a user to assist in achieving a consistent tool orientation and accurate depth.

In various implementations, the gauge apparatus may be implemented by measuring a first offset of the orientation data and the depth data aligned with a first target region of a patient. In operation, the first offset associated with the first target may be designated by a user by actuating a first input of a user interface of the gauge apparatus when the tool accessory of the surgical hand tool is aligned with the first target according to the surgical procedure and corresponding plan. The activation of the first input may trigger a controller of the gauge apparatus to capture the orientation data and the depth data detected by the orientation sensor and the depth sensor to “zero out” or offset the orientation data and depth data based on the alignment of the surgical tool with the first target. With the first offset measured, the gauge apparatus may continue to track the device orientation and the tool depth of the surgical hand tool and the tool accessory relative to the first offset. By tracking the orientation and depth, the gauge apparatus may provide user feedback via a display indicating the tool orientation and the tool depth relative to the first offset. The orientation information and depth feedback demonstrated on the display may then be viewed by a user or surgeon to guide the surgical procedure.

In various implementations, the controller of the gauge apparatus may be configured to store a plurality of offsets corresponding to multiple targets associated with the surgical procedure. The offsets may correspond to the orientation and depth of the tool accessory when aligned with each of the corresponding targets of the patient anatomy. In operation, each of the offsets may be selectively stored and recalled throughout the operation of the surgical tool to provide both orientation and depth guidance based on the corresponding offsets measured as discussed herein. Accordingly, the gauge apparatus may be implemented to assist in a variety of surgical procedures.

These and other features, objects and advantages of the present disclosure will become apparent upon reading the following description thereof together with reference to the accompanying drawings.

### BRIEF DESCRIPTION OF THE DRAWINGS

FIG. 1 is a side perspective view of a surgical hand tool demonstrating a gauge apparatus;

FIG. 2 is a side perspective view of a surgical hand tool demonstrating an exemplary operation of a gauge apparatus;

FIG. 3 is a flow chart demonstrating a method for operating a gauge apparatus for a surgical hand tool;

FIG. 4 is a schematic diagram demonstrating a display of a gauge apparatus providing orientation and depth feedback corresponding to the operation of a surgical hand tool;

FIG. 5 is a rear perspective view demonstrating the assembly of a housing and locking collar of a gauge apparatus in connection with a surgical hand tool;

FIG. 6A is a side perspective view of a locking collar for a gauge apparatus in an extended configuration being assembled in connection with a surgical hand tool;

FIG. 6B is a side perspective view demonstrating a locking collar in a locked configuration with a housing of the gauge apparatus in connection with the surgical hand tool; and

FIG. 7 is a block diagram demonstrating a control structure of the gauge apparatus as discussed herein.

### DETAILED DESCRIPTION

In the following description, reference is made to the accompanying drawings, which show specific implementations that may be practiced. Wherever possible, the same reference numbers will be used throughout the drawings to refer to the same or like parts. It is to be understood that other implementations may be utilized and structural and functional changes may be made without departing from the scope of this disclosure.

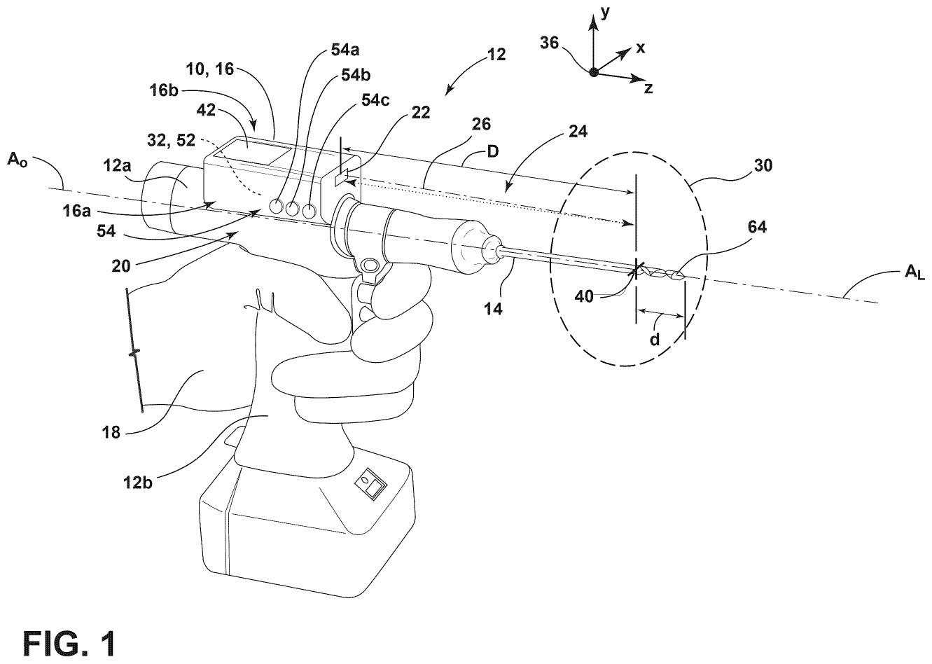

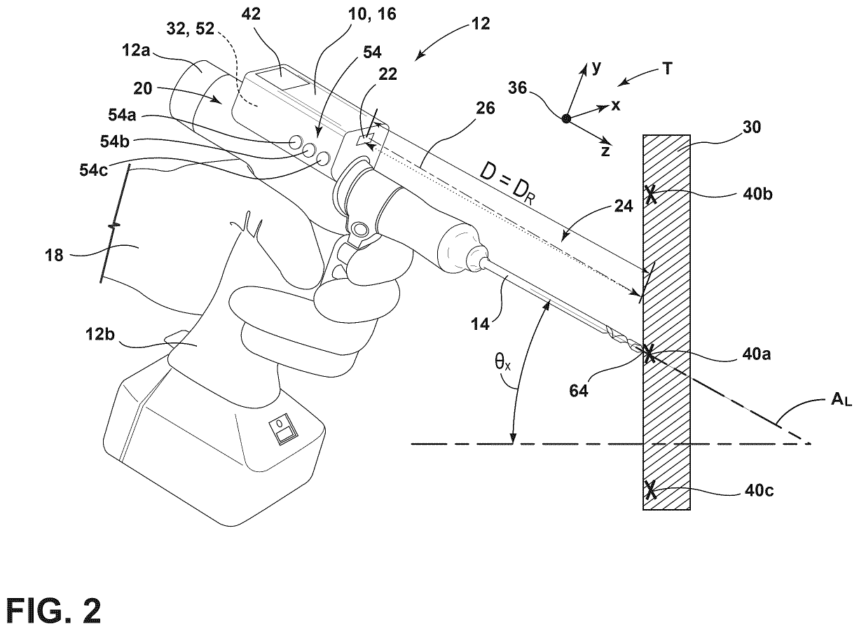

As generally demonstrated in FIGS. 1 and 2, the disclosure provides for a gauge apparatus 10 for a surgical tool 12 or surgical hand tool. As previously described in the background section, the accuracy of operation of various surgical tools may vary based on the skill and experience of the user or surgeon. In particular, the use of surgical tools 12 as described herein may provide for considerable flexibility in the execution of various procedures but may also contribute to variations in cutting or driving paths of surgical tools. For example, surgical plans for various procedures may rely on proper alignment and accurate execution for various driving, cutting, and/or resecting steps necessary to complete procedures. In operation, the gauge apparatus 10 may provide alignment and depth feedback to a surgeon or user of various types of surgical tools 12 that may ensure a path of a tool accessory 14 (e.g., a cutting tool, driving tool, etc.) may be consistently aligned with a planned orientation while the hand tool is engaged with a target of the patient anatomy. Additionally, the gauge apparatus 10 may provide for feedback demonstrating a tool depth of the tool accessory relative to the target throughout the operation of the surgical hand tool 12.

As demonstrated in the exemplary figures, the tool accessory 14 of the surgical tool 12 may correspond to a cutting tool in the form of a drill. However, in various implementations, the tool accessory may correspond to a variety of cutting tools and/or driving tools that may be operated via a variety of rotary, oscillating, reciprocating, or other forms of motion associated with the operation of a surgical tool 12. Examples of the tool accessory 14 as cutting tools may include various forms of reamers, drills, endmills, saws, shavers, burrs, etc. Further, implementations of the tool accessory 14 as driving tools may include one or more driver bits, pin drivers, socket drivers, or similar tools that may be implemented for surgical procedures. Accordingly, the disclosure may be generally applicable to the application of various surgical tools 12 and corresponding tool accessories 14 to facilitate a broad range of surgical procedures.

As shown in FIG. 1, the gauge apparatus 10 is incorporated in a housing 16 in connection with a body 12a of the surgical tool 12. In the example of the tool 12 implemented as a driver, the gauge apparatus 10 may be in connection with the body 12a adjacent to a handle portion 12b that may be engaged by a hand of a user 18 to maneuver the tool 12. The housing 16 may be positioned in connection with a motor enclosure 20 housing a motor (not shown) that may define an operating axis A_{O} of the surgical tool 12. In this configuration, a depth sensor 22 incorporated within the housing 16 may be substantially aligned with the operating axis A_{O} of the surgical tool 12 and, in the example shown, aligned parallel with a longitudinal axis A_{L} of the tool accessory 14. In this configuration, a detection field 24 defining a path of a detection emission 26 (e.g., laser, ultrasonic, etc.) may be output from the depth sensor 22 aligned with or parallel to the longitudinal axis A_{L} of the tool accessory 14. As later discussed in reference to the operation of the depth sensor 22, the alignment of the detection field 24 with the longitudinal axis A_{L} may ensure that changes in a distance “D” between the depth sensor 22 and an object 30 (e.g., a patient anatomy) may be representative of changes in a depth “d” of the tool accessory 14 engaging or protruding into or through the object 30. In this configuration, changes in the distance D reported by the depth sensor 22 may be representative of the depth d of the tool accessory 14 extending into the object 30.

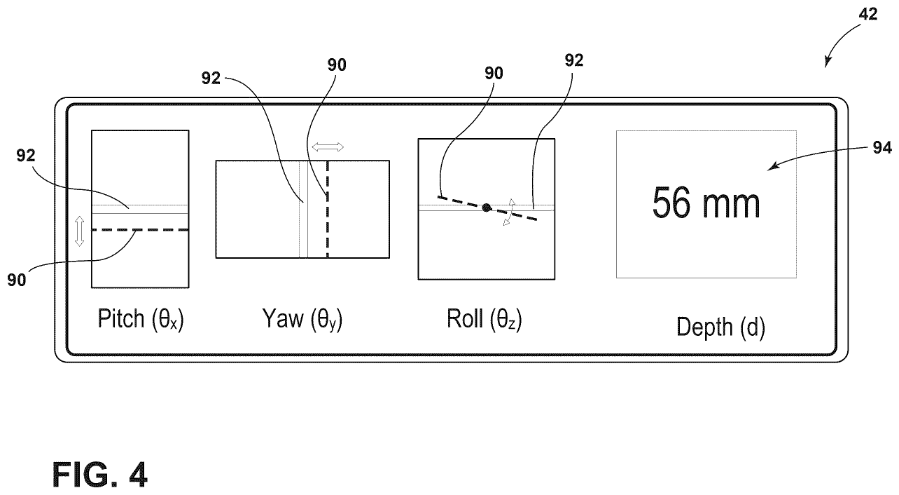

In addition to the depth sensor, the gauge apparatus 10 may further include an orientation sensor 32, which may be incorporated within the housing 16. The orientation sensor 32 may be configured to detect orientation data identifying a device orientation 36 of the surgical tool 12 relative to a prevailing force (e.g., gravity). As shown, the device orientation 36 may be tracked by the orientation sensor 32 about a plurality of axes (e.g., X, Y, Z), which may correspond to a pitch θ_{X}, a yaw θ_{Y}, and a roll θ_{Z} as depicted in FIG. 4. In operation, orientation data reported by the orientation sensor 32 may be monitored relative to one or more offsets that may correspond to aligned orientations of the tool accessory 14 with one or more targets 40 associated with a surgical procedure. In this way, the orientation data provided by the orientation sensor 32 may provide for meaningful feedback to a user via a display device 42 to assist in operation of the surgical hand tool 12.

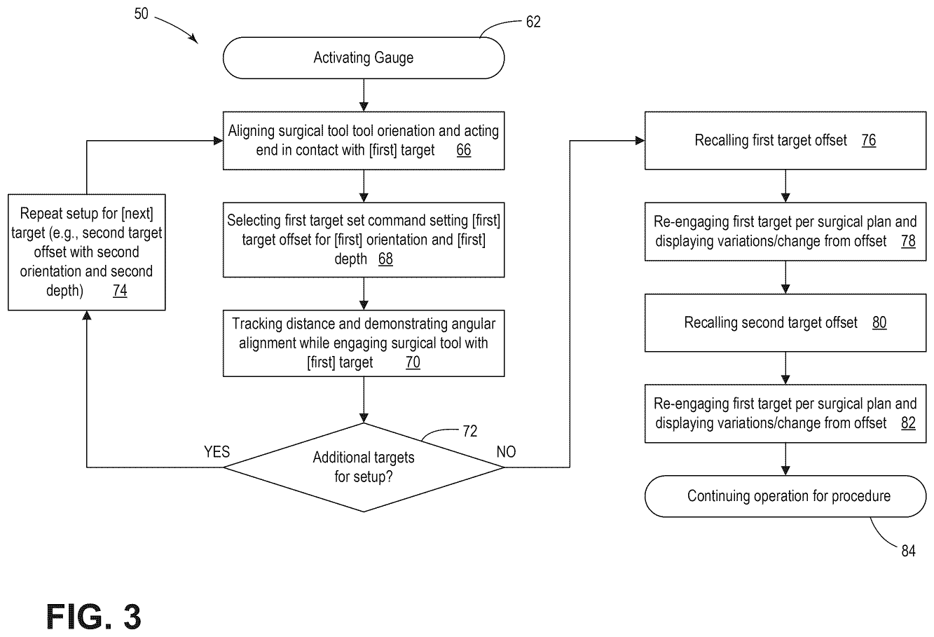

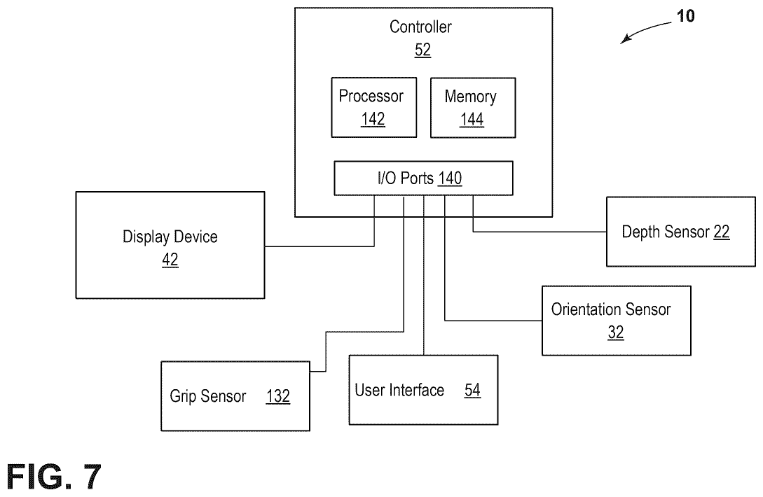

In an exemplary operation, as later detailed in reference to the method 50 demonstrated in FIG. 3, a controller 52 (see FIG. 7) of the gauge apparatus 10 may be configured to selectively set and recall a plurality of orientation offsets and/or depth offsets identifying an orientation of the surgical tool 12 and a corresponding distance D between the depth sensor 22 and the object 30 with the tool accessory 14 aligned with a target 40. As shown in FIG. 2, the tool 12 is aligned with a first target 40a at a first orientation designated as a trajectory T at reference distance DR. Once positioned according to the surgical plan, the user 18 of the tool 12 may set a first offset to the trajectory T at reference distance DR. Based on variations from the first offset, the controller 52 may track and update the display 42 to track the relative orientation of the surgical tool 12 and depth d of the tool accessory 14 aligned with the first target 40a. Following the programming of the first offset with the first target 40a, the user may similarly program and/or recall additional offsets aligned with multiple targets 40 (e.g., second target 40b, third target 40c) associated with the surgical procedure. In this way, the gauge apparatus 10 may provide for the setting or designation of the trajectory T in response to an input to a user interface 54, thereby storing the reference distance DR reported by the depth sensor 22 and the orientation 36 reported by the orientation data for multiple targets 40. By demonstrating variations from the orientation 36 and distance D associated with each of the offsets on the display 42, the gauge apparatus 10 may demonstrate variations in the orientations (e.g., θ_{X}, θ_{Y}, and θ_{Z}) and the depth d of the tool accessory 14 in relation to the corresponding target 40.

Referring now to FIGS. 2-4, the operation of the gauge apparatus 10 is discussed in reference to the method 50 of FIG. 3. As shown in FIG. 2, following the activation of the gauge apparatus 10 in step 62, the surgical tool 12 may be aligned in a tool orientation defining the trajectory T with an acting end or distal end 64 of the tool accessory 14 aligned with and in contact with a first target 40a (66). With the device orientation 36 aligned to the target 40, a first orientation and first depth associated with the first target 40a may be assigned and stored to a memory of the controller 52 in response to a first input 54a to the user interface 54 (68). In the example shown, the device orientation 36 associated trajectory defined by the rotation angles θ_{X}, θ_{Y}, and θ_{Z} as well as the distance D equal to the reference distance DR may be saved to the memory of the controller 52 as a first offset. With the first offset stored to the memory of the controller 52, the method 50 may demonstrate variations in the orientation 36 resulting from changes relative to the trajectory T set to the first offset. Additionally, variations in the distance D resulting from penetration over the depth d into the target 40 of the object 30 may be tracked (70). As previously discussed, feedback demonstrating variations in the rotation angles θ_{X}, θ_{Y}, and θ_{Z} and the depth d may be updated and demonstrated on the display device 42, as shown in FIG. 4.

Following the set up and storing of the device orientation 36 and distance D associated with the first target 40a, the method 50 may continue to determine if additional targets need to be set up and stored to the memory of the controller 52 in step 72. If additional targets (e.g., second target 40b, third target 40c, etc.) are planned for the procedure, the method 50 may continue to step 74 to repeat steps 66-70 for the second target 40b, third target 40c, etc. In each case, the user of the surgical tool 12 and gauge apparatus 10 may assign a corresponding offset (e.g., second offset, third offset, etc.) by selecting a corresponding input (e.g., second input 54b, third input 54c, etc.) on the user interface 54 in repeated step 68. In this way, the device orientation 36 and the depth d may be assigned to corresponding offsets aligned with each of the targets 40a, 40b, 40c, etc. As further discussed in reference to steps 76-82, the ongoing steps of the method 50 may provide for each of the offsets of the device orientation 36 aligned with the corresponding targets 40a, 40b, 40c, etc. to be recalled selectively throughout operation of the method 50. In this way, a user of the surgical tool 12 may recover the original alignment corresponding to the offsets to ensure that the positioning of the tool accessory 14, including the device orientation 36 and the distance D, may be repeatedly aligned to each of the corresponding targets 40 in a highly accurate and repeatable manner.

In steps 76-82, the method may continue by recalling the first target offset in step 76 or the second target offset in step 80. In conjunction with recalling the offsets for the corresponding targets 40a, 40b, a user of the surgical tool 12 may realign or reengage the acting or distal end 64 of the tool accessory 14 with each of the corresponding targets 40a, 40b in steps 78 and 82. With the offsets corresponding to the targets 40a, 40b selectively recalled, the controller 52 may update the differences of the rotation angles θ_{X}, θ_{Y}, and θ_{Z} and the change in the distance D and/or resulting depth d on the display device 42, as further discussed in reference to FIG. 4. Throughout the operation of the gauge apparatus 10 and the surgical tool 12, the offsets may be updated for additional targets 40 and selectively recalled in response to corresponding selections to the user interface 54 (84). Though discussed in reference to recalling the first offset and the second offset in steps 76 and 80, it shall be understood that a user of the gauge apparatus 10 may selectively recall and/or store the offsets corresponding to various targets in any order throughout the use of the surgical tool 12.

Referring now to FIG. 4, the alignment feedback and depth indication output by the controller 52 are shown on the display device 42. In the example shown, the display device may provide for user feedback in the form of variations in the pitch θ_{X}, yaw θ_{Y}, roll θ_{Z}, and depth d. However, it shall be understood that the specific configuration and feedback information demonstrated on the display device 42 may be configured to demonstrate the rotation angles θ_{X}, θ_{Y}, and θ_{Z} and/or the distance D or depth d associated with the operation of the surgical tool 12 in any combination. Accordingly, the controller 52 may provide for the display device 42 to provide feedback for the most meaningful rotation angles θ associated with the device orientation 36 and depth d or distance D associated with the operation of the tool accessory 14 according to the critical alignment characteristics associated with each of the targets 40.

As shown in FIG. 4, the relative alignment of the device orientation 36 associated with each of the rotation angles θ_{X}, θ_{Y}, and θ_{Z} may be demonstrated as a level indicator 90. The offset corresponding to the device orientation 36, as identified by the orientation sensor 32 during the setup procedure exemplified by the method 50, may be depicted on the display device 42 as a set-point indicator 92. The relative angle of the surgical tool 12 deviating from the offset may be visually identified by a user by comparing the position of the level indicator 90 to the set-point indicator 92 on the display device 42. In the example shown, the pitch θ_{X} is demonstrated with the level indicator 90 varying upward or downward relative to the set-point indicator 92. The yaw θ_{Y} is demonstrated with the level indicator 90 shifting left to right relative to the set-point indicator 92. The roll θ_{Z} is demonstrated with the level indicator 90 rotating relative to the set-point indicator 92 about the Z-axis, which may be aligned with the longitudinal axis A_{L} of the tool accessory 14. In this way, the deviation of the device orientation 36 about each of the rotation angles θ_{X}, θ_{Y}, θ_{Z} as detected by the orientation sensor 32 may be depicted by the variations and the level indicator 90 relative to the set-point indicator 92. Such feedback may allow the user of the surgical tool 12 to accurately and repeatedly align the tool accessory 14 with the trajectory T of each of the targets 40 to achieve highly repeatable and accurate alignment.

Still referring to FIG. 4, the depth d of the tool accessory 14 as detected by the depth sensor 22 may be depicted as a depth indicator 94. The depth indicator may demonstrate variations in the distance D resulting in the depth d of the tool accessory 14 penetrating into the object 30 at each of the targets 40. As shown, the depth indicator 94 may correspond to a numeric measurement standard that may vary in magnitude or direction in response to changes in the distance D between the depth sensor 22 and the surface of the object 30. The numeric indication of the depth d or distance D is displayed in millimeters (mm) and may be calibrated to accurately detect changes in the distance D to a precision of plus or minus 1 mm, 0.1 mm, or better depending on the type of device implemented for the depth sensor 22. Accordingly, the display device 42 of the gauge apparatus 10 may provide highly beneficial feedback to the user of the surgical tool 12 throughout operation to improve the accuracy and repeatability of various procedures.

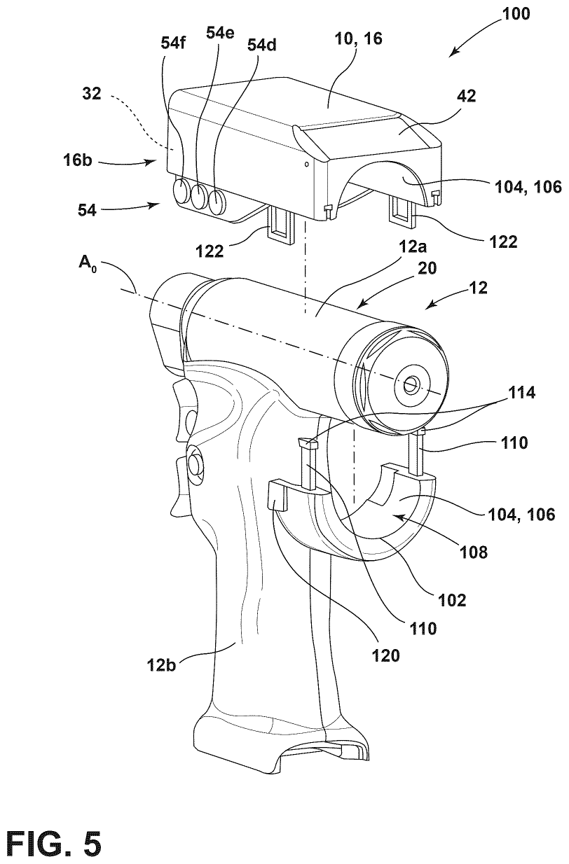

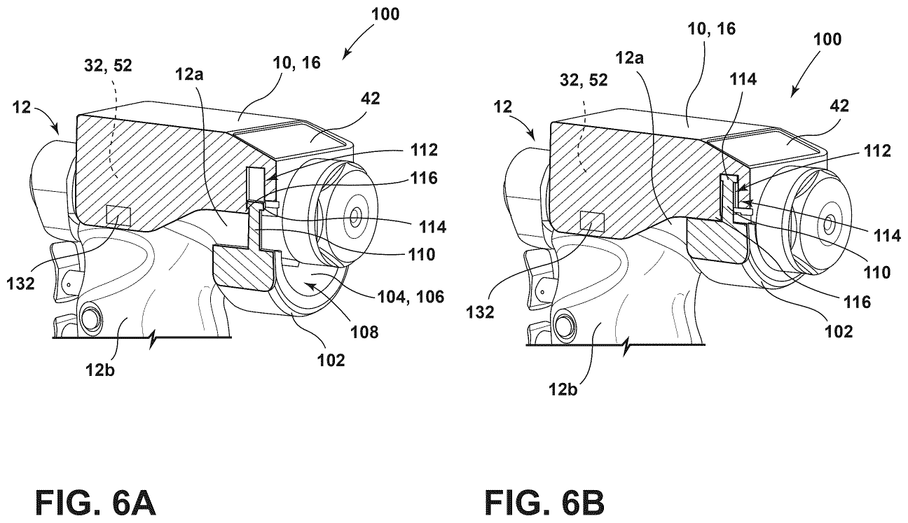

Referring now to FIGS. 5, 6A, and 6B, the gauge apparatus 10 may correspond to a detachable accessory 100 comprising a locking collar 102 configured to selectively connect the housing 16 of the gauge apparatus 10 to the body 12a of the surgical tool 12. As demonstrated in FIG. 5, the locking collar 102 and housing 16 may form a mounting surface 104 having an interior profile 106 defining an opening 108 configured to engage an exterior contour shape of the body 12a of the surgical tool 12. In the example shown, the corresponding portion of the body 12a of the surgical tool 12 is cylindrical in shape and extends parallel to and approximately equidistant about the operating axis A_{O} of the surgical tool 12. In this configuration, the engagement of the locking collar 102 to the housing 16 and resulting assembly of the mounting surface 104 to the body 12a of the surgical tool 12 may align the detection field 24 of the depth sensor 22 and the device orientation 36 with the operating axis A_{O} of the surgical tool 12. In this configuration, the detection field 24 and the detection emission 26 of the depth sensor 22 may be aligned substantially parallel with the longitudinal axis A_{L} of the tool accessory 14 as shown in FIGS. 1 and 2. Accordingly, the position and orientation of the housing 16 of the gauge apparatus 10 may be aligned to the operating axis A_{O} of the surgical tool 12 via the alignment of the mounting surface 104 and fixed engagement provided by the locking collar 102 in connection with the housing 16.

As further demonstrated in FIGS. 6A and 6B, in some implementations, the locking collar 102 may engage the housing 16 via at least one retention tab 110. In the example shown, retention tabs 110 are incorporated on opposing sides of the locking collar 102 and slidably engage corresponding retention slots 112 on opposing sides of the housing 16. As demonstrated in FIG. 6A, each of the retention tabs 110 may include a retention feature 114, which may correspond to a protrusion or variation in a longitudinal profile shape of the retention tab 110. In the example shown, the retention features 114 correspond to T-shaped protrusions that engage a corresponding detent 116 formed at a distal extent of the retention tabs 110 that are engaged by the retention features 114 at the extended position of the locking collar 102 relative to the housing 16. The retention of the locking collar 102 to the housing 16 may allow the user 18 to connect the gauge apparatus 10 to the body 12a of the tool 12 with a single hand without interrupting the operation of the tool 12.

In operation, the retention tabs 110 may slide within the retention slots 112, allowing the locking collar 102 to enclose about the body 12a of the surgical tool 12. The locking engagement of the collar 102 to the body 12a may be provided by a user gripping the housing 16 and the locking collar 102 and sliding the mounting surface 104 into contact with the body 12a of the tool 12. With the mounting surface 104 of the detachable accessory 100 engaged with the body 12a, a locking tab 120 (FIG. 5) may engage a receiving bracket 122, thereby securing the locking collar 102 to the housing 16. In the example shown, the receiving bracket 122 is formed by a portion of the housing 16, and the locking tab is formed on an exterior surface of the locking collar 102. However, it shall be understood that the retention tabs 110, locking tabs 120, receiving brackets 122, and other portions of a connection interface 124 may be formed in connection with the housing 16 and/or the locking collar 102.

As best demonstrated in FIGS. 1 and 5, the user interface 54 may be formed along one or more side portions 16a, 16b of the housing 16, which may be on opposing sides of the body 12a of the surgical tool 12. In some implementations, the user interface 54 and the corresponding inputs (first input 54a, second input 54b, third input 54c, etc.) may be formed on both sides of the housing 16 on opposing sides of the body 12a of the surgical tool 12, allowing for ambidextrous operation by the digits of the user 18 grasping a top surface of the gauge apparatus 10 with a nonworking hand. Such a configuration may allow the user or surgeon to readily engage the handle 12b with a working hand and control the operation of the gauge apparatus 10 via the user interface 54 with the nonworking hand. Accordingly, the positions of the inputs 54a, 54b, etc. of the user interface 54 may be provided on opposing sides of the housing 16, allowing for left-handed or right-handed operation of the gauge apparatus 10.

An additional benefit of providing the inputs on opposing sides of the gauge apparatus 10 may be to allow the selective configuration of the left-handed or right-handed operation, such that the inputs (e.g., 54a, 54b, 54c, 54d, 54e, 54f) on each side 16a, 16b of the gauge apparatus 10 provide for functions that may vary depending upon a handedness configuration setting. For example, if a user is left-handed, as demonstrated in FIG. 1, a right hand may be used to readily engage the inputs on the second side 16b of the housing 16. With the user interface configured for left-handed usage, the inputs 54d, 54e, 54f (FIG. 5) on the second side 16b of the housing 16 may be configured to activate primary operations of first operations (e.g., setting and recalling the offsets for the targets 40) while inputs 54a, 54b, 54c on the first side 16a (FIG. 1) may be configured to control secondary or second/auxiliary commands for the gauge apparatus 10. Alternatively, in a right-handed configuration, the inputs 54a, 54b, 54c on the first side 16a may be configured to control first operations while the inputs 54d, 54e, 54f on the second side 16b may be configured to provide the second operations assigned by the controller 52. In this way, the handedness configuration of the gauge apparatus 10 may provide for differing configurations and corresponding updated control mapping of each set of inputs 54a-54c, 54d-54f on opposing sides 16a, 16b of the housing 16. Such operation may ensure the gauge apparatus 10 provides for flexible operation regardless of the handedness of the user.

In some implementations, the handedness of the user may be detected by the gauge apparatus 10. For example, in addition or alternative to providing the handedness setting as a device setting accessed and controlled by the user interface 54, the gauge apparatus 10 and/or the surgical tool 12 may include one or more sensors (e.g., proximity sensors, contact sensors, etc.) that may detect the side on which the hand of the user 18 engages the surgical tool 12. For example, as illustrated in FIGS. 6A and 6B, a handedness or grip sensor 132 may be incorporated on either or both sides 16a, 16b of the housing 16 or body 12a of the gauge apparatus 10 or the surgical tool 12. The grip sensor 132 may identify the side 16a, 16b on which the handle 12b of the surgical tool 12 is grasped by the hand of the user 18. In response to identifying the side on which the surgical tool 12 is held and operated, the controller 52 may adjust the mapping and control configuration associated with the inputs 54a-54c and 54d-54f on the opposing sides 16a, 16b of the surgical tool 12.

Referring now to FIG. 7, a block diagram of the gauge apparatus 10 is shown demonstrating the controller 52 comprising a plurality of communication or input/output ports 140 in communication with the sensors 22, 32 as well as the display device 42 and the user interface 54. In various implementations, the controller 52 may provide for a variety of programmable operations that may be enabled by a processor 142 implementing various routines stored in a memory 144. In various implementations, the processor 142 may correspond to a wide variety of programmable devices that may be implemented as microprocessors, microcontrollers, application specific integrated controllers (ASICs), or similar devices. The memory 144 may correspond to various forms of non-transitory, machine readable media that may include read-only memory, programmable read-only memory, electronically erasable programmable read-only memory, etc. Accordingly, the processor 142 and memory 144 may be implemented with a variety of devices depending on the specific application of the gauge apparatus 10 to support the operation of the gauge apparatus 10.

The depth sensor 22 is demonstrated in communication with the controller 52 via the communication ports 140. The depth sensor 22 may be implemented with a variety of sensory devices that may detect the range or distance D of the object 30 along the detection field 24. In the example shown, the depth sensor 22 corresponds to a time-of-flight sensor that monitors the distance D associated with the time-of-flight of the detection emission 26 as a laser or light emission. Though discussed as a time-of-flight sensor, various sensors that may detect the distance D may be implemented including ultrasonic sensors, optical sensors, etc. The orientation sensor 32 is also demonstrated in communication with the controller 52 via the communication ports 140. In various implementations, the orientation sensor 32 may correspond to a multi access accelerometer, an inertial measurement unit, gyroscope, or various orientation-sensing devices that may be implemented alone or in combination. Accordingly, the sensors 22, 32 associated with the gauge apparatus 10 may be implemented in a variety of ways.

In addition to the sensors 22, 32, the user interface 54, grip sensor 132, and the display device 42 may also be in communication with the controller 52 via the communication ports 140. As discussed in reference to various control routines, the display device 42 may provide for visual feedback identifying variations in the pitch θ_{X}, yaw θ_{Y}, roll θ_{Z}, and depth d. As previously discussed, the display of the rotation angles θ_{X}, θ_{Y}, and θ_{Z} may be selectively activated depending on the application or the type of tool accessory 14 implemented. For example, a roll θ_{Z} of the surgical tool 12 may not provide meaningful feedback if the tool accessory 14 is a drill or driver. However, the roll θ_{Z} of the surgical tool 12 may be highly relevant in aligning the operation of a saw. Accordingly, the controller 52 may provide for the display device 42 to provide feedback for the most meaningful rotation angles θ associated with the device orientation 36 and depth d or distance D associated with the operation of the tool accessory 14.

According to some aspects of the disclosure, a gauge apparatus for a surgical tool comprises a tool accessory in the form of a cutting tool or driving tool. The gauge apparatus includes an orientation sensor configured to detect orientation data identifying a device orientation of the surgical tool about a plurality of axes; a depth sensor having a detection field extending along a longitudinal axis of the tool accessory, the depth sensor configured to measure depth data indicating a tool depth along the longitudinal axis; a display device; and a controller. The controller is configured to set a first offset at a first orientation and a first depth of the tool accessory of the surgical tool in response to a first input received with the surgical tool, wherein the first offset is assigned based on the orientation data and the depth data at a first time of the first input; track the device orientation and the tool depth of the surgical tool relative to the first offset; display the device orientation relative to the first orientation of the first offset on the display device based on the orientation data; and display the tool depth relative to a first target on the display device in response to the depth data measured relative to the first depth.

According to various aspects, the disclosure may implement one or more of the following features or configurations in various combinations:
the depth sensor is a non-contact sensor operable to detect a time-of-flight of a proximity signal output from an emitter;
the orientation sensor comprises an accelerometer configured to detect the device orientation as two or more of a yaw, a pitch, and a roll of the surgical tool relative to the first orientation;
the roll is about the longitudinal axis of the tool accessory;
the device orientation is displayed as a level indicator that shifts along or about one of the plurality of axes relative to a set-point indicator demonstrating the first orientation of the first offset;
the first offset is based on the orientation data identified at the first orientation and the depth data identified at the first depth with the tool accessory of the surgical tool aligned with the first target;
a housing forming a user interface in connection with the surgical tool, wherein the user interface comprises a plurality of user inputs including the first input;
the orientation sensor, the depth sensor, and the display device are connected to the surgical tool by the housing;
the housing forms a locking collar that engages a body of the surgical tool in a locked position that secures the locking collar in connection to the housing about the body via at least one locking tab;
the locking collar comprises at least one retention tab that interconnects the housing to the locking collar, wherein the retention tab slidably engages the housing adjusting the locking collar from an extended position to the locked position;
at least one retention tab slides along a retention slot to the extended position, and wherein an opening formed between the locking collar and the housing in the extended position receives a motor enclosure of the surgical tool;
an interior mounting surface formed by the housing and the locking collar engages corresponding exterior surfaces of the housing and the motor enclosure surgical tool in the locked position;
the cutting tool comprises one of a reamer, a drill, an end mill, a saw, a shaver, and a burr;
the driving tool comprises one of a driver bit, a pin driver, and a socket driver;
the controller is further configured to set a second offset at a second orientation and a second depth of the tool accessory of the surgical tool in response to a second input received with the surgical tool aligned with a second target, wherein the second offset is assigned based on the orientation data and the depth data at a second time of the second input;
the controller is further configured to track the device orientation relative to the surgical tool relative to the second offset;
the controller is further configured to display the device orientation relative to the second orientation of the second offset on the display device based on the orientation data; and
displaying a depth change relative to the second target on the display device in response to the depth data measured relative to the second depth; and/or
controller is further configured to selectively recall the first offset in response to the first input and the second offset in response to the second input.

According to another aspect of the disclosure, a method for tracking an orientation and depth of a tool accessory of a surgical tool comprises locating a surgical tool in a first orientation aligned with a first target and capturing orientation data identifying a device orientation of the surgical tool about a plurality of axes and identifying a distance to a surface of an object forming the first target along a longitudinal axis of the tool accessory. In response to receiving a first input, the method sets a first offset at a first orientation and a first depth of the tool accessory of the surgical tool with the surgical tool aligned with the first target; tracks the device orientation and the distance to the surface relative to the first offset; displays the device orientation relative to the first orientation of the first offset on the display device based on the orientation data; and displays a depth change relative to the first target on the display device in response to the distance measured relative to the first depth.

According to various aspects, the disclosure may implement one or more of the following features or configurations in various combinations:
in response to receiving a second input, setting a second offset at a second orientation and a second depth of the tool accessory of the surgical tool with the surgical tool aligned with the second target;
tracking the device orientation and the distance to the surface relative to the second offset;
displaying the device orientation relative to the second orientation of the second offset on the display device based on the orientation data;
displaying a depth change relative to the second target on the display device in response to the distance measured relative to the second depth; and/or
selectively recalling the first offset in response to the first input and the second offset in response to the second input.

According to yet another aspect of the disclosure, a gauge apparatus for a surgical cutting or driving tool is disclosed. The gauge apparatus comprises a housing forming a user interface selectively connected to the surgical tool the housing in connection with an orientation sensor, a depth sensor, and a controller. The orientation sensor is configured to detect orientation data identifying a device orientation about a plurality of axes, and the depth sensor monitors a detection field extending along a longitudinal axis of the tool accessory. The depth sensor is configured to measure depth data indicating a tool depth along the longitudinal axis. A display device is in communication with the controller. The controller is configured to set a first offset at a first orientation and a first depth of the tool accessory of the surgical tool in response to a first input to the gauge apparatus. The controller is further configured to track the device orientation and the tool depth of the surgical tool relative to the first offset. Based on the orientation data, the controller outputs the device orientation relative to the first orientation of the first offset to the display device. In response to the depth data measured relative to the first depth, the controller outputs the tool depth relative to a first target to the display device.

It will be understood that any described processes or steps within described processes may be combined with other disclosed processes or steps to form structures within the scope of the present device. The exemplary structures and processes disclosed herein are for illustrative purposes and are not to be construed as limiting.

It is also to be understood that variations and modifications can be made on the aforementioned structures and methods without departing from the concepts of the present device, and further it is to be understood that such concepts are intended to be covered by the following claims unless these claims by their language expressly state otherwise.

The above description is considered that of the illustrated embodiments only. Modifications of the device will occur to those skilled in the art and to those who make or use the device. Therefore, it is understood that the embodiments shown in the drawings and described above are merely for illustrative purposes and not intended to limit the scope of the device, which is defined by the following claims as interpreted according to the principles of patent law, including the Doctrine of Equivalents

1. A gauge apparatus for a surgical tool comprising a tool accessory in the form of a cutting tool or driving tool, the gauge apparatus comprising:
an orientation sensor configured to detect orientation data identifying a device orientation of the surgical tool about a plurality of axes;
a depth sensor having a detection field extending along a longitudinal axis of the tool accessory, the depth sensor configured to measure depth data indicating a tool depth along the longitudinal axis;
a display device; and
a controller configured to:
set a first offset at a first orientation and a first depth of the tool accessory of the surgical tool in response to a first input, wherein the first offset is assigned based on the orientation data and the depth data at a first time of the first input;
track the device orientation and the tool depth of the surgical tool relative to the first offset;
display the device orientation relative to the first orientation of the first offset on the display device based on the orientation data; and
display the tool depth relative to a first target on the display device in response to the depth data measured relative to the first depth.

2. The gauge apparatus according to claim 1, wherein the depth sensor is a non-contact sensor operable to detect a time-of-flight of a proximity signal output from an emitter.

3. The gauge apparatus according to claim 1, wherein the orientation sensor comprises an accelerometer configured to detect the device orientation as two or more of a yaw, a pitch, and a roll of the surgical tool relative to the first orientation.

4. The gauge apparatus according to claim 3, wherein the roll is about the longitudinal axis of the tool accessory.

5. The gauge apparatus according to claim 1, wherein the device orientation is displayed as a level indicator that shifts along or about one of the plurality of axes relative to a set-point indicator demonstrating the first orientation of the first offset.

6. The gauge apparatus according to claim 1, wherein the first offset is based on the orientation data identified at the first orientation and the depth data identified at the first depth with the tool accessory of the surgical tool aligned with the first target.

7. The gauge apparatus according to claim 1, further comprising a housing forming a user interface in connection with the surgical tool, wherein the user interface comprises a plurality of user inputs including the first input.

8. The gauge apparatus according to claim 7, wherein the orientation sensor, the depth sensor, and the display device are connected to the surgical tool by the housing.

9. The gauge apparatus according to claim 7, wherein the housing forms a locking collar that engages a body of the surgical tool in a locked position that secures the locking collar in connection to the housing about the body via at least one locking tab.

10. The gauge apparatus according to claim 9, wherein the locking collar comprises at least one retention tab that interconnects the housing to the locking collar, wherein the retention tab slidably engages the housing adjusting the locking collar from an extended position to the locked position.

11. The gauge apparatus according to claim 10, wherein the at least one retention tab slides along a retention slot to the extended position, and wherein an opening formed between the locking collar and the housing in the extended position receives a motor enclosure of the surgical tool.

12. The gauge apparatus according to claim 11, wherein an interior mounting surface formed by the housing and the locking collar engages corresponding exterior surfaces of the housing and the motor enclosure of the surgical tool in the locked position.

13. The gauge apparatus according to claim 1, wherein the cutting tool comprises one of a reamer, a drill, an end mill, a saw, a shaver, and a burr.

14. The gauge apparatus according to claim 11, wherein the driving tool comprises one of a driver bit, a pin driver, and a socket driver.

15. The gauge apparatus according to claim 1, wherein the controller is further configured to:
set a second offset at a second orientation and a second depth of the tool accessory of the surgical tool in response to a second input received with the surgical tool aligned with a second target, wherein the second offset is assigned based on the orientation data and the depth data at a second time of the second input;
track the device orientation and the tool depth of the surgical tool relative to the second offset;
display the device orientation relative to the second orientation of the second offset on the display device based on the orientation data; and
display a depth change relative to the second target on the display device in response to the depth data measured relative to the second depth.

16. The gauge apparatus according to claim 15, wherein controller is further configured to:
selectively recall the first offset in response to the first input and the second offset in response to the second input.

17. A method for tracking an orientation and depth of a tool accessory of a surgical tool, the method comprising:
locating a surgical tool in a first orientation aligned with a first target;
capturing orientation data identifying a device orientation of the surgical tool about a plurality of axes and identifying a distance to a surface of an object forming the first target along a longitudinal axis of the tool accessory;
in response to receiving a first input, setting a first offset at a first orientation and a first depth of the tool accessory of the surgical tool with the surgical tool aligned with the first target;
tracking the device orientation and the distance to the surface relative to the first offset;
displaying the device orientation relative to the first orientation of the first offset on the display device based on the orientation data; and
displaying a depth change relative to the first target on the display device in response to the distance measured relative to the first depth.

18. The method according to claim 17, further comprising:
in response to receiving a second input, setting a second offset at a second orientation and a second depth of the tool accessory of the surgical tool with the surgical tool aligned with the second target;
tracking the device orientation and the distance to the surface relative to the second offset;
displaying the device orientation relative to the second orientation of the second offset on the display device based on the orientation data; and
displaying a depth change relative to the second target on the display device in response to the distance measured relative to the second depth.

19. The method according to claim 17, further comprising:
selectively recalling the first offset in response to the first input and the second offset in response to the second input.

20. A gauge apparatus for a surgical cutting or driving tool, the gauge apparatus comprising:
a housing forming a user interface selectively connected to the surgical tool the housing in connection with an orientation sensor, a depth sensor, and a controller, wherein the orientation sensor is configured to detect orientation data identifying a device orientation about a plurality of axes, and the depth sensor having a detection field extending along a longitudinal axis of the tool accessory, the depth sensor configured to measure depth data indicating a tool depth along the longitudinal axis; and
a display device in communication with the controller, wherein the controller is configured to:
set a first offset at a first orientation and a first depth of the tool accessory of the surgical tool in response to a first input to the gauge apparatus, wherein the first offset is assigned based on the orientation data and the depth data at a time of the first input;
track the device orientation and the tool depth of the surgical tool relative to the first offset;
output the device orientation relative to the first orientation of the first offset based on the orientation data to the display device; and
output the tool depth relative to a first target to the display device in response to the depth data measured relative to the first depth.
